(19) United States
(12) Patent Application Publication
(10) Pub. No.: US 2026/0224357 A1
(43) Pub. Date: Aug. 6, 2026

(54) A PROTHESIS CONFIGURED TO PREVENT OR RESTRICT BILLOWING

(71) Applicant: Medtronic, Inc., Minneapolis, CA (US)

(72) Inventors: Amal ELGAMIL, Aliso Viejo, CA (US); Alexander D. CETNAR, Costa Mesa, CA (US)

(21) Appl. No.: 19/152,159

(22) PCT Filed: Jan. 30, 2024
(86) PCT No.: PCT/IB2024/050852

§ 371 Date: Jul. 30, 2025

Related U.S. Application Data
(60) Provisional application No. 63/443,817, filed on Feb. 7, 2023.

(51) Int. Cl. A61F 2/24; A61F 2/00; A61F 2/91

(52) U.S. Cl. CPC A61F 2/2418; A61F 2002/0081; A61F 2/91; A61F 2210/0014; A61F 2210/0076; A61F 2230/0093; A61F 2250/0056

(57) ABSTRACT

A prosthesis is provided having a radially expanded configuration and a radially compressed configuration. The prosthesis includes a frame including a plurality of side openings, and a skirt coupled to an interior of the frame and at least partially covering at least one side opening of the plurality of side openings. The skirt comprises a pressure-relief arrangement including at least one fluid passage through the area of the skirt at least partially covering the at least one side opening that is configured to prevent or restrict billowing of an area of the skirt extending over the at least one side opening.

### CROSS-REFERENCE TO RELATED APPLICATIONS

This application claims the benefit of U.S. application Ser. No. 63/443,817, filed Feb. 7, 2023, the entire content of which is incorporated herein by reference.

### FIELD

The present teachings relate to a prosthesis, and in particular to a valve prosthesis.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position. Known heart valve prosthesis

The present disclosure relates to improvements in a heart valve prosthesis to ensure that the heart valve prosthesis has a low profile for transcatheter delivery through a patient's vasculature.

### SUMMARY

According to a first aspect there is provided a prosthesis having a radially expanded configuration and a radially compressed configuration, the prosthesis comprising: a frame including a plurality of side openings; and a skirt coupled to an interior of the frame and at least partially covering at least one side opening of the plurality of side openings, wherein the skirt comprises a pressure-relief arrangement configured to prevent or restrict billowing of an area of the skirt extending over the at least one side opening, and wherein the pressure relief arrangement comprises at least one fluid passage through the area of the skirt at least partially covering the at least one side opening.

The pressure-relief arrangement is configured to enable some of a patient's blood to flow through the skirt to reduce the pressure applied to the skirt. This pressure relief reduces, restricts or prevents billowing of the skirt material that spans across the side opening of the frame of the valve prosthesis, as such billowing may undesirably result in contact between the skirt and the leaflets of the valve prosthesis after the valve prosthesis is deployed in situ. If the leaflets of the valve prosthesis contact the skirt during opening and closing in situ, such contact may cause early leaflet tissue abrasion as well as early skirt abrasion due to the undesired billowing of the skirt. Additionally, greater relative motion between the skirt and the frame may further induce early skirt abrasion. Early leaflet tissue abrasion and/or early skirt abrasion has a negative impact on the long-term durability of the valve prosthesis.

This reduction in billowing of the skirt has been found to enable a reduction in the number of frame struts needed to be used on the frame, which results in a lower profile of the prosthesis when in the radially compressed configuration for transcatheter delivery through a patient's vasculature.

In some embodiments, the at least one fluid passage may comprise at least one aperture extending through the skirt. For example, in some embodiments, each aperture may comprise a slit through the skirt or a cut out portion of the skirt. The use of slits and/or cut outs through the skirt facilitates the amount of pressure relief that is provided (e.g. through size and quantity of the apertures), whilst ensuring that sufficient flow and pressure is upheld to maintain normal functionality of the prosthesis.

In some embodiments, the skirt may comprise a plurality of apertures extending through the area of the skirt at least partially covering the at least one side opening. In some embodiments, the or each aperture may define an elongate axis that is arranged to be parallel, perpendicular, or angled relative to an axis extending between an inflow end and an outflow end of the prosthesis. In some embodiments, the or each aperture may be substantially rectangular, circular, or elliptical. In some embodiments, the skirt may comprise a fabric material and the or each aperture is woven or knitted into the skirt.

In some embodiments, the at least one fluid passage may be configured to permit fluid to flow in a radially inward direction through the skirt and to block fluid flow in a radially outward direction. In this way, the at least one fluid passage may act as a check valve, also known as a non-return valve. This prevents backflow of blood through the material of the skirt in an upstream direction.

In some embodiments, the pressure relief arrangement may comprise a moveable member, e.g. a moveable flap, associated with the at least one fluid passage and configured and arranged to selectively close the at least one fluid passage to prevent fluid flow through the skirt in a radially outward direction. This prevents backflow of blood through the material of the skirt in an upstream direction. In some embodiments, for example, the skirt may comprise a fabric material, and wherein the moveable member is woven or knitted into the skirt.

In some embodiments, the skirt may comprise a fabric material configured to prevent liquid from flowing therethrough, and wherein the at least one fluid passage is defined by a region where the fabric material is configured to coney fluid through the skirt in a radially inward direction. In some embodiments, for example, the skirt may comprise a hydrophobic or superhydrophobic fabric material configured to prevent liquid from flowing therethrough, and wherein the at least one fluid passage is defined by a region where the fabric material comprises a reduced hydrophobicity relative to the hydrophobic fabric that is configured convey fluid through the skirt in a radially inward direction, optionally wherein the at last one fluid flow channel is defined by a region containing a reduced number of hydrophobic groups.

In some embodiments, the region may be defined by an area of the fabric of increased wettability relative to the fabric material. In some embodiments, the region may comprise a gradient in the hydrophobicity of the fabric material such that the hydrophobicity of the fabric reduces from a radially outer side to a radially inner side of the fabric material. In some embodiments, the at least one fluid passage may be configured to permit directional transport through the skirt in a radially inward direction, and to prevent flow through the at least one fluid passage in a radially outward direction.

In some embodiments, the skirt may comprise a substantially tubular body and first and second substantially circular edges, and wherein the first and/or second edge comprises at least one region defining a curved recessed edge. Providing a curved upstream edge of the skirt reduces fabric volume, which helps to reduce provide a lower profile of the prosthesis when in the radially compressed configuration. In some embodiments, for example, the at least one fluid passage may comprise at least one aperture extending through the skirt, said at least one aperture positioned at or adjacent to the curved recessed edge.

In some embodiments, the skirt may comprise a polymeric skirt material. In some embodiments, the polymeric skirt material may comprise one or more of PET, PTFE, and/or polyester.

In some embodiments, the at least one side opening of the plurality of side openings may be substantially diamond-shaped. For example, each of the plurality of side openings may be substantially diamond-shaped.

In some embodiments, the frame may comprise a plurality of crowns and a plurality of struts with each crown being formed between a pair of opposing struts, and wherein the plurality of side openings are defined by the plurality of crowns and the plurality of struts. In some embodiments, the frame may comprise a row of side openings extending around a perimeter thereof, and wherein the row comprises less than twelve side openings, optionally less than ten side openings, for example in the range four to ten side openings, for example in the range six to nine side openings. In some embodiments, the row may comprise three, six, or nine side openings.

In some embodiments, the skirt may be coupled to an inner surface or an outer surface of the frame, and wherein the skirt extends over fewer than all of the plurality of side openings of the frame.

In some embodiments, the frame may be formed from an expandable material. For example, the frame may be formed from a self-expanding material. In some embodiments, the self-expanding material may comprise a shape memory material, for example nitinol.

In some embodiments, the frame may comprise an inner frame and an outer frame, and wherein the plurality of side openings comprises a plurality of side openings on the inner frame and the skirt comprises an inner skirt coupled to the inner frame. In some embodiments, the outer frame may be connected to the inner frame and radially surrounds the inner frame, when the prosthesis is in the radially expanded configuration. In some embodiments, the plurality of side openings may comprise a plurality of side openings on the outer frame and the skirt comprises an outer skirt coupled to the outer frame.

In some embodiments, the prosthesis may be a heart valve prosthesis comprising a prosthetic valve component disposed within and secured to the inner frame, the prosthetic valve being configured to prevent blood flow in one direction to convey blood flow through a central lumen of the inner frame. In some embodiments, the heart valve prosthesis may be configured for placement within a mitral heart valve or tricuspid heart valve in situ.

### BRIEF DESCRIPTION OF THE DRAWINGS

Embodiments will now be described with reference to the accompanying drawings, in which:

FIG. 1 is a perspective view of a prosthesis in accordance with an embodiment of the disclosure;

FIG. 2 is an atrial or inflow end view of the prosthesis shown in FIG. 1;

FIG. 3 is a ventricular or outflow end view of the prosthesis shown in FIG. 1;

FIG. 4 is a perspective view of an inner frame of the prosthesis of FIG. 1 with a prosthetic valve component secured therein;

FIG. 5 is an enlarged side view of a side opening of the inner frame of FIG. 2;

FIG. 6 is a perspective atrial end view of the prosthesis shown in FIG. 1;

FIG. 7 is an atrial end view of the prosthesis shown in FIG. 1;

FIG. 8 is a schematic cross-sectional view of the prosthesis shown in FIG. 1;

FIG. 9 is a side view of an inner frame of a prosthesis according to an embodiment;

FIG. 10 is a side view of an inner frame of a prosthesis according to an embodiment;

FIG. 11 is a side view of an inner frame of a prosthesis according to an embodiment; and

FIG. 12 is a side view of a prosthesis in accordance with an embodiment of the disclosure;

### DETAILED DESCRIPTION OF CERTAIN EMBODIMENTS

The human heart includes right and left atriums, and right and left ventricles. Disposed between the left atrium and left ventricle is the mitral valve. As left atrial pressure increases above that of left ventricle, the mitral valve opens and blood passes into the left ventricle. The mitral valve is formed from a pair of leaflets having distal edges that meet so as to close the mitral valve. Each leaflet is attached to an annular region of the heart structure known as the valve annulus. Disposed between the right atrium and the right ventricle is the tricuspid valve. As right atrial pressure increases above that of right ventricle, the tricuspid valve opens and blood passes into right ventricle. The tricuspid valve is formed from three leaflets having distal edges that meet so as to close the tricuspid valve. Each leaflet is attached to an annular region of the heart structure known as the valve annulus. In addition to mitral valve and tricuspid valve, the heart includes the aortic valve and the pulmonary valve. The aortic valve permits one-way flow of blood from the left ventricle to the aorta. The pulmonary valve permits one-way flow of blood from right ventricle to pulmonary artery. Each of the aortic valve and the pulmonary valve are formed from three leaflets having distal edges that meet so as to close the respective valve. Each leaflet is attached to an annular region of the heart structure known as the valve annulus. The present teachings relate to a heart valve prosthesis for replacing diseased and/or damaged heart valves. In particular, the teachings relate to a skirt-reinforcement member for supporting or reinforcing a skirt that spans across a side opening of a frame of the prosthesis, e.g. the heart valve prosthesis.

The illustrated embodiments relate to a transcatheter valve prosthesis in the form of a heart valve prosthesis configured for placement within a mitral heart valve or a tricuspid heart valve. In alternative embodiments, skirt-reinforcement members described herein may be utilized with a transcatheter heart valve configured for placement within a pulmonary, aortic, mitral, or tricuspid valve, or may be utilized with a transcatheter valve prosthesis configured for placement within a venous valve or within other body passageways where it is deemed useful. It will be understood that the embodiments of skirt-reinforcement members described herein may be utilized with any prosthesis having a frame and a skirt for which reinforcement thereof is desirable to limit billowing, and is not intended to be limited to a prothesis having an inner frame and an outer frame coupled to and radially surrounding the inner frame.

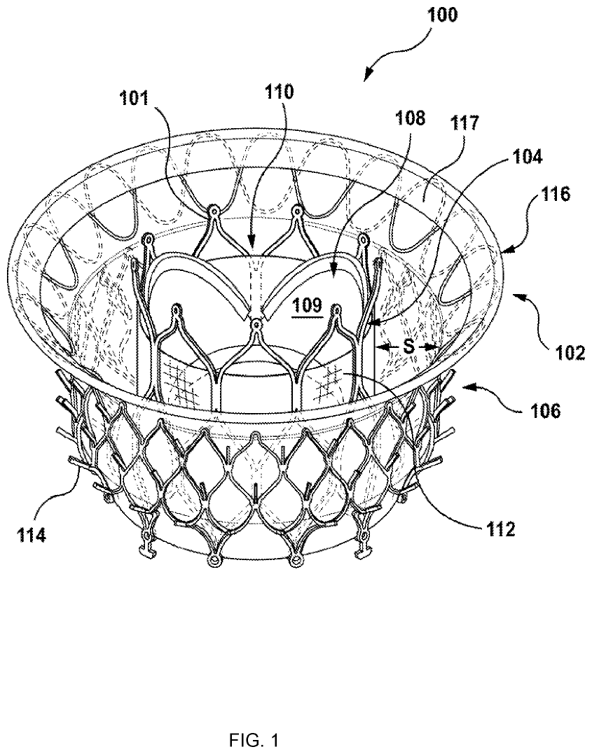

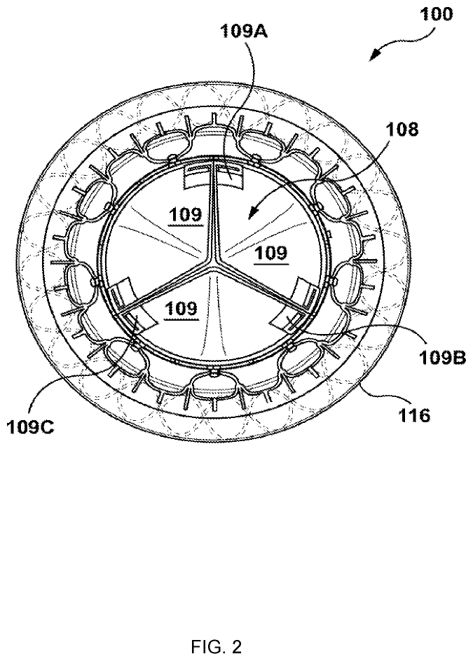

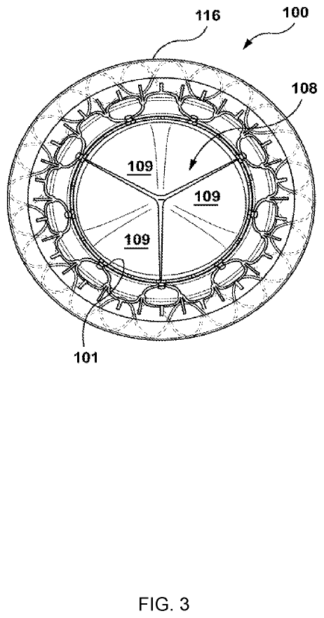

Referring to FIGS. 1 to 3, a transcatheter valve prosthesis is illustrated and is indicated generally at 100. The transcatheter valve prosthesis 100 is configured to be radially compressed into a reduced-diameter crimped configuration for delivery within a vasculature (not shown) and to return to an expanded, deployed configuration, as is illustrated in Figure FIG. 1. Stated another way, the transcatheter valve prosthesis 100 has a crimped configuration for delivery within a vasculature and an expanded configuration for deployment within a native heart valve. When in the crimped configuration, the transcatheter valve prosthesis 100 has a low profile suitable for delivery to and deployment within a native heart valve via a suitable delivery catheter that may be tracked to the deployment site of the native heart valve of a heart via any one of a transseptal, retrograde, or transapical approach.

The transcatheter valve prosthesis 100 includes a stent or frame 102. The frame 102 may be considered to be a dual frame. The frame 102 includes a valve support or inner frame 104. The frame 102 includes an anchor element or outer frame 106. The inner frame 104 is at least partially surrounded (e.g. radially) by and coupled to the outer frame 106, when the prosthesis 100 is in the radially expanded configuration. The prosthesis 100 includes a prosthetic valve component 108 including at least one leaflet disposed within and secured to the frame 102.

The inner frame 104 may be tubular or cylindrical shape. The inner frame 104 defines a central lumen 110 from an inflow end 101 to an outflow end 103 thereof. The inner frame 104 is configured to support the prosthetic valve component 108 therein, which will be described in more detail below. In an embodiment, the outflow end 103 has a diameter that is substantially the same as a diameter of the inflow end 101.

The prosthesis 100 includes an inner skirt 112. The inner skirt 112 is coupled to the inner frame 104. In the embodiment shown, the inner skirt 112 is coupled to an inner surface of the inner frame 104 to line a portion thereof. Alternatively, the inner skirt 112 may be coupled to an outer surface of the inner frame 104. The inner skirt 112 may be a natural or biological material such as pericardium or another membranous tissue such as intestinal submucosa. The inner skirt may be formed from a polymeric material. The inner skirt 112 may be a fabric, for example a low-porosity woven or braided fabric. Examples of such materials are polyethylene terephthalate (PET), polyester, Dacron fabric, or PTFE. The inner skirt 112 creates fluid boundaries of a one-way fluid passage when attached to the inner frame 104 and combined with a valve. In one embodiment, the inner skirt 112 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. In some embodiments, the inner skirt 112 may be provided with a coating of a surface coating material. Put another way, the inner skirt 112 may have a layer of a coating material on a surface thereon. The surface coating material may comprise a polymeric material. The surface coating material may comprise one or more of: PTFE; polyurethane; silicone; and hydrogels.

The outer frame 106 may be tubular or cylindrical shape. The outer frame 106 functions as an anchor for the transcatheter valve prosthesis 100 to secure its deployed position within a native annulus. The outer frame 106 is configured to engage heart tissue at or below an annulus of a native heart valve, such as an annulus of a native mitral valve or native tricuspid valve. At the inflow end 101 of the inner frame 104, the outer frame 106 is radially spaced a distance S from the inner frame 104 to mechanically isolate the inflow end 101 of the inner frame 104 from the outer frame 106. The outer frame 106 includes one or more cleats or prongs 114 that extend outward from an exterior side thereof to engage heart tissue. In another embodiment, the outer frame 106 may employ barbs, spikes, or other tissue fixation mechanisms for engaging heart tissue. In some embodiments, in cross-section, or viewed along its longitudinal axis, the outer frame 106 may be D-shaped, cylindrical, elliptical, or irregularly shaped. The outer stent may also have an angled or irregular side profile. For example, the outflow end of the outer frame 106 may be angled across one or both of its proximal or distal edges. This can be useful, for example, in that it can provide additional frame-profile options that may reduce obstruction of surrounding anatomy, such as the left or right ventricular outflow tracts (LVOT and RVOT, respectively) if they prosthetic valve is implanted in either the mitral or tricuspid annulus.

The inner frame 104 and/or the outer frame 106 may be formed from a self-expanding material, for example a shape memory material. The shape memory material may be a shape memory polymer, or a shape memory alloy, such as Nitinol™. Any portion of the frame 102 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected to provide the transcatheter heart valve prothesis 100 to be configured to be compressed into a reduced-diameter crimped configuration for transcatheter delivery to a native valve, whereby release from a delivery catheter returns the prosthesis to an expanded, deployed configuration. In alternative embodiments, the frame (i.e. the inner and/or outer frame) may be formed from an expandable material. In such embodiments, the frame may be expanded by an expanded means, such as a balloon.

The prosthesis 100 includes an optional brim or rim element 116. Other embodiments may not include a brim or rim element. Here, the brim 116 extends outwardly from an upstream end of the outer frame 106. The brim element 116 includes a brim frame. In the embodiments shown, the brim frame includes overlapping, 180° out of phase sinusoidal wire forms, although it will be appreciated that any suitable brim frame may be used. The brim frame is attached to the outer frame 106 by an outer skirt 117. The outer skirt 117 may be any suitable biocompatible material, for example a low-profile fabric used in bioprosthetic implants, such as woven polyethylene terephthalate (PET) fabric. The brim element 116 may act as an atrial retainer, if present. In order to provide this function, the brim element 116 may be configured to engage tissue above a native annulus, such as a supra-annular surface or some other tissue in the left atrium, to thereby inhibit downstream migration of a prosthetic heart valve 100.

The prosthetic valve component 108 of the transcatheter valve prosthesis 100 is capable of regulating flow therethrough via valve leaflets that may form a replacement valve. In the illustrated embodiments, the valve component 108 is shown has having three leaflets, although a single leaflet or bicuspid leaflet configuration may alternatively be used. When deployed in situ, the prosthetic valve component 108 in a closed state is configured to block blood flow in one direction to regulate blood flow through the central lumen 110 of the inner frame 104.

The prosthetic valve component 108 includes valve leaflets 109, e.g., two or three valve leaflets 109. The valve leaflets 109 are disposed to coapt within an upstream portion of the inner frame 104. Leaflet commissures 109A, 109B, 109C of the valve leaflets 109 are secured within a downstream portion of the inner frame 104, such that the valve leaflets 109 open during diastole. Leaflets 109 are attached along their bases to the inner frame 104, for example, using sutures or a suitable biocompatible adhesive. Adjoining pairs of leaflets 109 are attached near or to one another at their lateral ends to form leaflet commissures 109A, 109B, 109C. The orientation of the leaflets 109 within the inner frame 104 depends upon on which end of the transcatheter valve prosthesis 100 is the inflow end and which end of the transcatheter valve prosthesis 100 is the outflow end, thereby ensuring one-way flow of blood through the transcatheter valve prosthesis 100.

The valve leaflets 109 may be attached to the inner skirt 112. The valve leaflets 109 may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

For delivery, the transcatheter valve prosthesis 100 is radially compressed into a reduced-diameter crimped configuration onto a delivery system for delivery within a vasculature. Example delivery systems may include an inner shaft that receives the transcatheter valve prosthesis 100 on a distal portion thereof and an outer sheath or capsule that may be configured to retain the transcatheter valve prosthesis 100 on the distal portion of the inner shaft during delivery. It may compressively retain the transcatheter valve prosthesis if the prosthesis is made of a self-expanding material. Stated another way, the outer sheath or a capsule, in some embodiments, surrounds and constrains the transcatheter valve prosthesis 100 in the radially compressed or crimped configuration. In other embodiments, such as those where the prosthesis is balloon-expandable, the sheath or capsule need not compressively retain the prosthesis, but may still cover or protect the prosthesis during delivery.

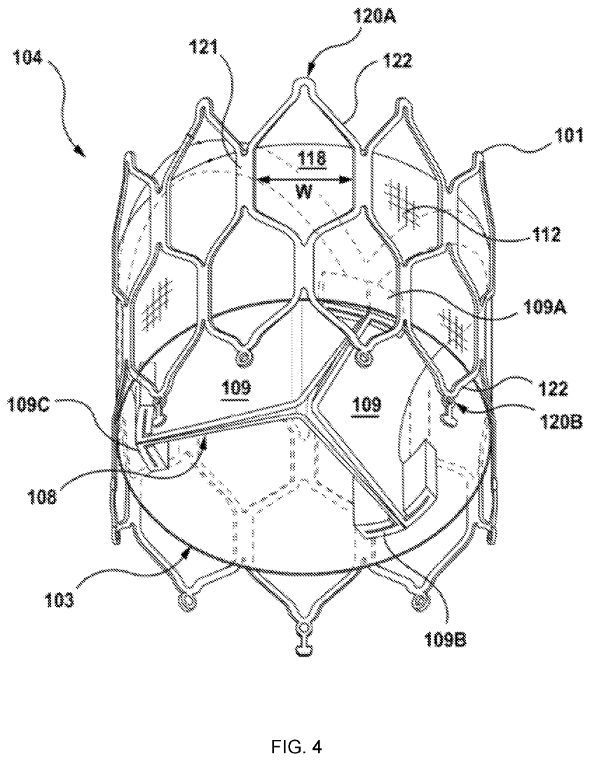

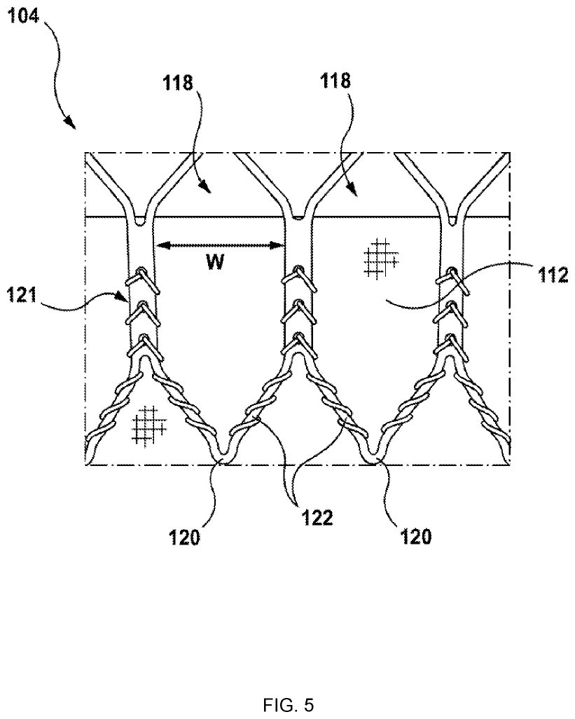

Referring to FIGS. 4 and 5, the inner frame 104 includes a plurality of side openings 118. The inner frame 104 may include a row of, or other embodiments of such as scattered or non-row, side openings 118 around a circumference thereof. In some embodiments, a row of side openings may include more or fewer than 12 side openings, and, for example fewer than 12 or fewer than 10 side openings. The role of side openings they include 13 or more side openings. In some embodiments, the inner frame 104 may include 3 to 11 side openings, 4 to 10 side openings, or 6 to 9 side openings in the row.

The inner frame 104 includes a plurality of crowns 120 and a plurality of struts 122 with each crown 120 being formed between a pair or more of the opposing struts 122. Each crown 120 is a curved segment or bend extending between opposing struts 122. The plurality of side openings 118 are defined by edges of the plurality of crowns 120 and the plurality of struts 122. In an embodiment, the plurality of side openings 118 may be substantially diamond or hexagon shaped. The inner frame 104 includes a plurality of nodes 121. A node 121 is defined as a region where two crowns of the plurality of crowns 120 within the inner frame 104 meet or connect.

The inner skirt 112 is attached to an inner surface of the inner frame 104 around a circumference thereof. The inner skirt 112 spans across or extends over a plurality of side openings 118 of the plurality of side openings 118. In some embodiments, it will be appreciated that the inner skirt 112 may only extend partially over the side openings 118. In some embodiments the inner skit 112 may extend over fewer than all of the plurality of side openings 118. Notably, as is illustrated, it is not required that the inner skirt 112 extend over the full opening of each side opening 118 and/or that the inner skirt 112 extend over each of the plurality of openings. Rather, the inner skirt 112 may span or cover only a portion of the side opening 118.

A series of endmost inflow crowns 120A are formed at the inflow end 101 of the inner frame 104. A series of endmost outflow crowns 120B are formed at the outflow end 103 of the inner frame 104. In an embodiment, the inflow end 101 of the inner frame 104 has a total of nine endmost inflow crowns 120A around a circumference thereof. In some embodiments, the outflow end 103 of the inner frame 104 has a total of nine endmost inflow crowns 120B around a circumference thereof. The inflow end 101 of the inner frame 104 includes a row of side openings 118 around a circumference thereof, and the row has a total of nine side openings 118. The outflow end 103 of the inner frame 104 includes a row of side openings 118 around a circumference thereof, and the row has a total of nine side openings 118. In another embodiment hereof (not shown), each of the inflow end 101 and the outflow end 103 of the inner frame 104 has between 3 and 11, or 4 to 10 side openings, or 6 to 9 side openings. In some embodiments, only a single row of side openings may be provided, or three or more rows of side openings may be provided. In some embodiments, each row may include three, six, or nine side openings.

In an embodiment, width W a width W of the side openings 118 is between 1/24 and ⅙ of the circumference of the inner frame 104, or stated another way, between 4% and 16% of the circumference of the inner frame 104. By increasing the width of the side openings 118, a lesser amount of material is required for the inner frame 104 such that a lower profile may be achieved when the inner frame 104 is crimped into a radially compressed configuration for delivery. For example, the dual frame 102 of some embodiments includes both the inner frame 104 and the outer frame 106 compressed around a common length. It can be a challenge to reduce the profile of the transcatheter valve prosthesis 100 in the crimped or radially compressed configuration in these and other embodiments. A challenge with reducing the profile of any such embodiments is that, in the crimped or radially compressed configuration, the incompressible material of the frame 102 imparts high compressive forces on the soft tissue material of the leaflets 109. Compressive forces may alter the integrity of the leaflets 109, thereby impacting the long-term durability of the transcatheter valve prosthesis 100. However, increasing the width W of the side openings 118 provides a reduction of the incompressible material of the dual frame 102, thereby enabling a lower profile in the crimped or radially compressed configuration.

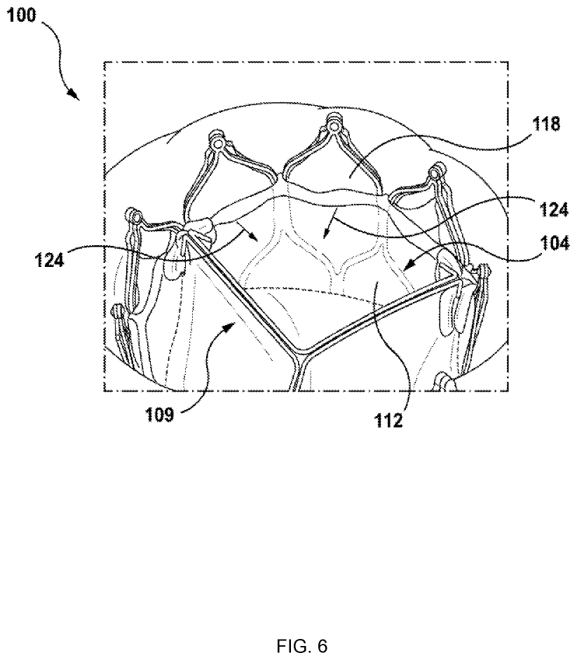

Reducing the incompressible material of the frame 102 means that the inner skirt 112 spans a longer distance between nodes 121 or between struts 122. Stated another way, increasing the width of the side openings 118 increases the amount of material of the inner skirt 112 that spans across the side openings 118. Thus, a greater amount of material of the inner skirt 112 is unattached to or unsupported by the inner frame 104. Referring now to FIGS. 6A and 6B, when an increased amount of material of the inner skirt 112 spans across the side openings 118, there is an increased chance of the inner skirt 112 billowing or moving radially inwards towards the leaflets 109 as indicated by directional arrows 124. The inner skirt 112 may billow during valve opening and closing in situ, and the leaflets 109 may contact the inner skirt 112. Such billowing may undesirably result in contact between the inner skirt 112 and the leaflets 109 of the transcatheter valve prosthesis 100. If the leaflets 109 of the transcatheter valve prosthesis 100 contact the inner skirt 112 during opening and closing, such contact may cause early leaflet tissue abrasion as well as early skirt abrasion. Additionally, the greater relative motion between the inner skirt 112 and the inner frame 104 may further induce early skirt abrasion.

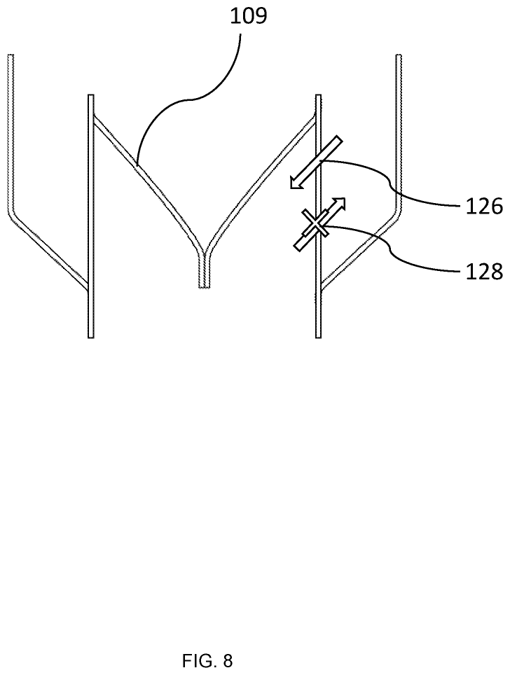

Referring now to FIG. 8, the prosthesis 100 is configured to prevent or restrict billowing of an area of the skirt 112 extending over, or at least partially covering, the at least one side opening. The skirt includes a pressure-relief arrangement configured to prevent or restrict billowing of an area of the skirt 112 extending over, or at least partially covering, the at least one side opening. Put another way, the pressure relief arrangement is configured to limit the radial motion of the skirt material. In this way, the pressure-relief arrangement minimizes risk of damage to both the inner skirt 112 and the leaflets 109. The pressure-relief arrangement includes at least one fluid passage through the area of the skirt 112 at least partially covering the at least one side opening 118. The pressure-relief arrangement is configured to enable some of a patient's blood to flow through the skirt 112 to reduce the pressure applied to the skirt 112. This pressure relief reduces or prevents billowing of the skirt material that spans across the side opening 118 of the frame 102 of the prosthesis 100.

It will be appreciated that the at least one fluid passage may be configured to permit fluid to flow in a radially inward direction 126 through the skirt 112 and to prevent fluid flow in a radially outward direction 128. In this way, the at least one fluid passage acts as a check valve, also known as a non-return valve. This arrangement prevents backflow of blood through the material of the skirt 112 in an upstream direction.

As has been discussed above, in some embodiments the skirt 112 may be formed from a fabric material. In some embodiments, the fabric material of the skirt 112 may be configured to prevent liquid from flowing therethrough, and wherein the at least one fluid passage through the fabric skirt 112 may be defined by a region where the fabric material is configured to coney fluid through the skirt 112 in a radially inward direction. Put another way, each area of the skirt may be configured to permit directional transport through the skirt 112 in a radially outward direction, and to prevent flow through the at least one fluid passage in a radially inward direction.

The skirt 112 may be formed from a hydrophobic material, for example superhydrophobic material, that is configured to prevent liquid from flowing therethrough. It will be appreciated that the fabric of the skirt 112 may be naturally hydrophobic, or may be coated and/or treated so as to be hydrophobic. The at least one fluid passage through the skirt 112 may be defined by a region where the fabric material has a reduced hydrophobicity relative to the hydrophobic fabric of the rest of the skirt 112. The area of reduced hydrophobicity may be that is configured convey fluid through the skirt 112 in a radially inward direction. The fabric, and in particular the at least one fluid passage through the fabric, may be defined by a gradient in hydrophilicity (e.g., from hydrophobic character to hydrophilic character) of at least one, e.g. a plurality, of areas of the skirt 112 along a direction from a radially outer side to a radially inner side of the skirt 112. The gradient in the hydrophobicity of the fabric material may be configured such that the hydrophobicity of the fabric reduces from a radially outer side to a radially inner side of the fabric material. The at least one fluid passage may be configured to permit directional transport through the skirt in a radially inward direction, and to prevent flow through the at least one fluid passage in a radially outward direction. The gradient in hydrophilicity or hydrophobicity (e.g., from hydrophobic character to hydrophilic character) may result from a gradient in concentration of hydrophobic groups. In this way, the fabric may be considered to define areas having a wettability gradient through the fabric. Put another way, the fabric may define areas of increased wettability relative to the rest of the fabric material.

It will be appreciated that the areas through the fabric skirt material described above may be formed by plasma treatment via a patterned mask to create the fluid passage or passages through the skirt 112. It will be appreciated that any suitable treatment may be used to one or more of the areas. In some embodiments, the areas may be provided over substantially of the skirt 112, or may only be provided in the areas of the skirt extending over the at least one side opening 118.

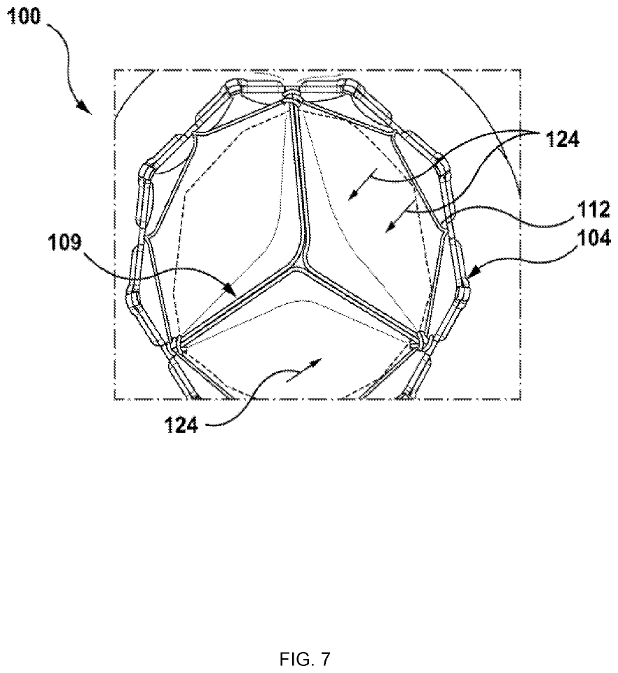

As has been discussed above, the transcatheter valve prosthesis 100 includes a stent or frame 102. The frame 102 may be considered to be a dual frame having a valve support or inner frame 104 and an anchor element or outer frame 106. In the embodiment illustrated in FIGS. 1 to 8, the outer frame 106 is coupled or connected to the inner frame 104 at or near to the outflow end 103 of the inner frame 104. It will be appreciated that in alternative embodiments, the outer frame 106 may be connected to the inner frame 104 at any suitable location, such as at or near the inflow end 101 of the inner frame 104 at a point between the inflow and outflow ends 101, 103.

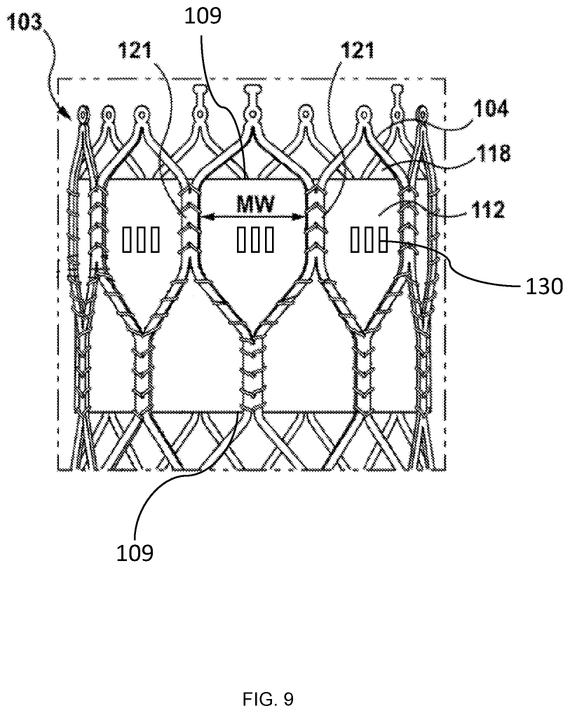

Referring now to FIGS. 9 to 11, embodiments of the pressure-relief arrangement are illustrated incorporating at least one, e.g. a plurality of, apertures 130 extending through the skirt 112 so as to define at least one fluid passage through the skirt 112. It will be appreciated that the pressure-relief arrangements illustrated in FIGS. 9 to 11 may be incorporated on the prosthesis embodiments illustrated in FIGS. 1 to 8 and 12, or any prosthesis having a frame and a skirt for which it is desirable to limit billowing.

In the embodiments shown, each aperture 130 is provided in the form of a slit through the skirt 112 or a cut out portion of the skirt 112. In embodiments where the skirt 112 is formed from a fabric material, the or each aperture 130 may be woven or knitted into the skirt 112. It will be appreciated that the size, shape, quantity and/or orientation of the or each cut-out or slit may be adjusted to suit the pressure-relief needs of the particular application, whilst ensuring that sufficient flow and pressure is upheld to maintain normal functionality of the prosthesis 100.

The skirt 112 is provided with three apertures 130 extending through each area of the skirt 112 that at least partially covers a side opening 118 of the frame 102 (i.e. the inner frame in this embodiment). It will be appreciated that each area extending over a side opening 118 may be provided with one, two, four or any suitable number of apertures 130. It will also be appreciated that the number of apertures at each side opening 118 may differ.

As is shown in the figures, each aperture 130 defines an elongate axis. In the embodiment shown, the elongate axis is arranged to be parallel to an axis extending between an inflow end and an outflow end of the skirt 112. Each aperture 130 is illustrated as being substantially rectangular. It will be appreciated that in alternative embodiments, the apertures(s) may be arranged perpendicular, or angled relative to the axis extending between an inflow end and an outflow end of the prosthesis and/or may be any suitable shape such as circular, or elliptical. It will be appreciated that in some embodiments, the size, shape, and/or orientation may be different for different apertures 130 through the skirt 112.

The pressure relief arrangement may include a moveable member, e.g. a moveable flap, 132 associated with the at least one fluid passage (i.e. the aperture 130). The moveable flap 132 is configured and arranged to selectively close the aperture 130 to prevent fluid flow through the skirt in a radially outward direction. In the embodiment, the moveable flap 132 is cut into the skirt 112. In embodiments where the skirt 112 is formed from a fabric material, the moveable member 132 may be woven or knitted into the skirt 112.

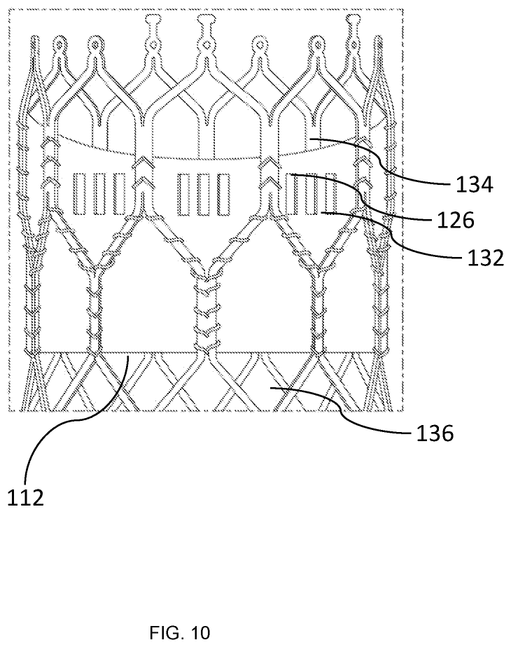

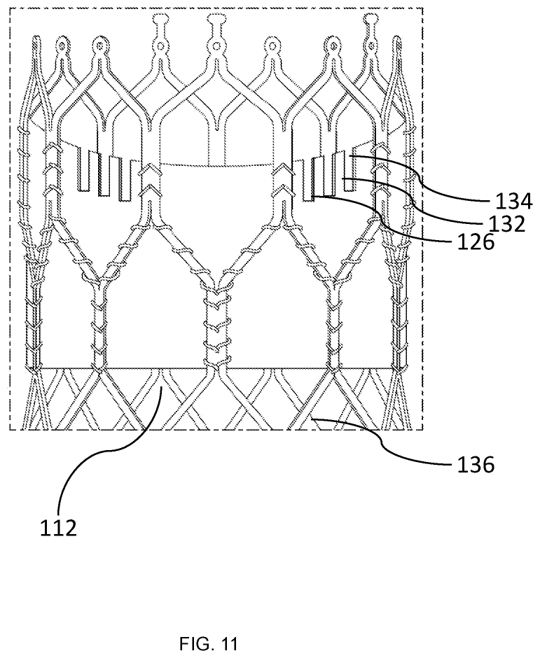

Referring now to FIGS. 10 and 11 embodiments of the skirt 112 are illustrated in which the skirt defines a curved or recessed edge 134.

The skirt 112 is substantially cylindrical and comprises a tubular body with first and second substantially circular edges 134, 136. The first and/or second edges 134, 136 may define at least one region having a curved recessed edge 134. In the embodiments shown, the skirt included two recessed regions extending around approximately half of the skirt 112. Providing a curved edge 134 of the skirt 112 reduces the total fabric volume, which provides a lower profile of the prosthesis 100 when in the radially compressed configuration. In some embodiments, the apertures 130 may be spaced apart from the upstream edge 134 (as illustrated in FIG. 10) or may be positioned at or adjacent to the curved recessed upstream edge 134 (as illustrated in FIG. 11). In some embodiments, the skirt 112 may include apertures at the curved edge 134 and also at a position spaced apart from said upstream edge.

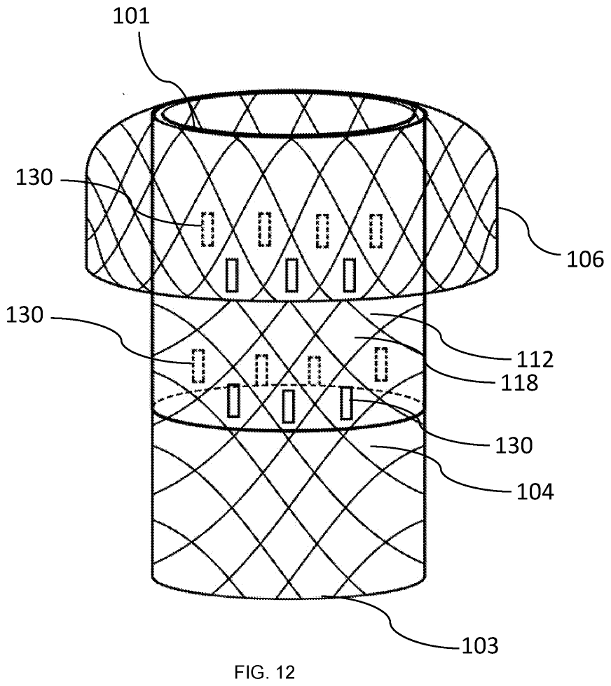

In some embodiments, for example as is illustrated in FIG. 12, the frame 102 may include an outer frame 106 that is defined by a portion of the frame 104 that is inverted so as to radially surround the inner frame 104. Put another way, the outer frame 106 may extend from an end of the inner frame 104 and be folded over so as to radially surround the inner frame 104 (e.g. such that the inner frame 104 is at least partially positioned within the outer frame 106). In the embodiment shown, the outer frame 106 extends from an inflow end 101 of the inner frame 104 but it will be appreciated that the outer frame 106 may extend from the outflow end 103 of the inner frame 104 in alternative embodiments. It will be appreciated that the prosthesis 100 illustrated in FIG. 12 may be configured to prevent or restrict billowing of an area of the skirt 112 extending over, or at least partially covering, the at least one side opening, as has been discussed with reference to the embodiment of FIGS. 1 to 11. It will also be appreciated that the prosthesis of the embodiment of FIG. 12 may include the curved recessed upstream edge discussed with reference to FIGS. 10 and 11.

It will be appreciated that the skirt-reinforcement arrangement described herein may be incorporated onto any prosthesis (e.g., a prothesis having a single frame or an inner frame or structure and an outer frame or structure coupled to and radially surrounding the inner frame or structure, or more than two frames or structures). It may be incorporated into any prosthetic having one or more skirts for which reinforcement or adaptation thereof is desirable to limit billowing, and it is not required that the structure, stent, or frame include a prosthetic valve component disposed therein.

Although the teachings have been described above with reference to one or more preferred embodiments, it will be appreciated that various changes or modifications may be made without departing from the scope as defined in the appended claims.

The following examples are illustrative of the techniques described herein.
Example 1: A prosthesis having a radially expanded configuration and a radially compressed configuration, the prosthesis comprising: a frame including a plurality of side openings; and a skirt coupled to an interior of the frame and at least partially covering at least one side opening of the plurality of side openings, wherein the skirt comprises a pressure-relief arrangement configured to prevent or restrict billowing of an area of the skirt extending over the at least one side opening, and wherein the pressure relief arrangement comprises at least one fluid passage through the area of the skirt at least partially covering the at least one side opening.
Example 2: The prosthesis according to Example 1, wherein the at least one fluid passage comprises at least one aperture extending through the skirt.
Example 3: The prosthesis according to Example 2, wherein each aperture comprises a slit through the skirt or a cut out portion of the skirt.
Example 4: The prosthesis according to claim 2, wherein the skirt comprises a plurality of apertures extending through the area of the skirt at least partially covering the at least one side opening.
Example 5: The prosthesis according to example 2, wherein the or each aperture defines an elongate axis that is arranged to be parallel, perpendicular, or angled relative to an axis extending between an inflow end and an outflow end of the prosthesis.
Example 6: The prosthesis according to example 2, wherein the or each aperture is substantially rectangular, circular, or elliptical.
Example 7: The prosthesis according to example 2, wherein the skirt comprises a fabric material and the or each aperture is woven or knitted into the skirt.
Example 8: The prosthesis according to example 1, wherein the at least one fluid passage is configured to permit fluid to flow in a radially inward direction through the skirt and to block fluid flow in a radially outward direction.
Example 9: The prosthesis according to example 1, wherein the pressure relief arrangement comprises a moveable member, e.g. a moveable flap, associated with the at least one fluid passage and configured and arranged to selectively close the at least one fluid passage to prevent fluid flow through the skirt in a radially outward direction.
Example 10: The prosthesis according to example 9, wherein the skirt comprises a fabric material, and wherein the moveable member is woven or knitted into the skirt.
Example 11: The prosthesis according to example 1, wherein the skirt comprises a fabric material configured to prevent liquid from flowing therethrough, and wherein the at least one fluid passage is defined by a region where the fabric material is configured to coney fluid through the skirt in a radially inward direction.
Example 12: The prosthesis according to example 1, wherein the skirt comprises a hydrophobic, for example superhydrophobic, fabric material configured to prevent liquid from flowing therethrough, and wherein the at least one fluid passage is defined by a region where the fabric material comprises a reduced hydrophobicity relative to the hydrophobic fabric that is configured convey fluid through the skirt in a radially inward direction, optionally wherein the at last one fluid flow channel is defined by a region containing a reduced number of hydrophobic groups.
Example 13: The prosthesis according to example 12, wherein the region is defined by an area of the fabric of increased wettability relative to the fabric material.
Example 14: The prosthesis according to example 12, wherein the region comprises a gradient in the hydrophobicity of the fabric material such that the hydrophobicity of the fabric reduces from a radially outer side to a radially inner side of the fabric material.
Example 15: The prosthesis according to example 12, wherein the at least one fluid passage is configured to permit directional transport through the skirt in a radially inward direction, and to prevent flow through the at least one fluid passage in a radially outward direction.
Example 16: The prosthesis according to example 1, wherein the skirt comprises a substantially tubular body and first and second substantially circular edges, and wherein the first and/or second edge comprises at least one region defining a curved recessed edge.
Example 17: The prosthesis according to example 16, wherein the at least one fluid passage comprises at least one aperture extending through the skirt, said at least one aperture positioned at or adjacent to the curved recessed edge.
Example 18: The prosthesis according to example 1, wherein the skirt comprises a polymeric skirt material.
Example 19: The prosthesis according to example 18, wherein the polymeric skirt material comprises one or more of PET, PTFE, and/or polyester.
Example 20: The prosthesis according to example 1, wherein the at least one side opening of the plurality of side openings is substantially diamond-shaped.
Example 21: The prosthesis according to example 20, wherein each of the plurality of side openings is substantially diamond-shaped.
Example 22: The prosthesis according to example 1, wherein the frame comprises a plurality of crowns and a plurality of struts with each crown being formed between a pair of opposing struts, and wherein the plurality of side openings are defined by the plurality of crowns and the plurality of struts.
Example 23: The prosthesis according to example 1, wherein the frame comprises a row of side openings extending around a perimeter thereof, and wherein the row comprises less than twelve side openings, optionally less than ten side openings, for example in the range four to ten side openings, for example in the range six to nine side openings.
Example 24: The prosthesis according to example 23, wherein the row comprises three, six, or nine side openings.
Example 25: The prosthesis according to example 1, wherein the skirt is coupled to an inner surface or an outer surface of the frame, and wherein the skirt extends over fewer than all of the plurality of side openings of the frame.
Example 26: The prosthesis according to example 1, wherein the frame is formed from an expandable material.
Example 27: The prosthesis according to example 26, wherein the frame is formed from a self-expanding material.
Example 28: The prosthesis according to example 26, wherein the self-expanding material comprises a shape memory material, for example nitinol.
Example 29: The prosthesis according to example 1, wherein the frame comprises an inner frame and an outer frame, and wherein the plurality of side openings comprises a plurality of side openings on the inner frame and the skirt comprises an inner skirt coupled to the inner frame.
Example 30: The prosthesis according to example 29, wherein the outer frame is connected to the inner frame and radially surrounds the inner frame, when the prosthesis is in the radially expanded configuration.
Example 31: The prosthesis according to example 29, wherein the plurality of side openings comprises a plurality of side openings on the outer frame and the skirt comprises an outer skirt coupled to the outer frame.
Example 32: The prosthesis according to example 1, wherein the prosthesis is a heart valve prosthesis comprising a prosthetic valve component disposed within and secured to the inner frame, the prosthetic valve being configured to prevent blood flow in one direction to convey blood flow through a central lumen of the inner frame.
Example 33: The prosthesis according to example 32, wherein the heart valve prosthesis is configured for placement within a mitral heart valve or tricuspid heart valve in situ.

What is claimed is:

1. A prosthesis having a radially expanded configuration and a radially compressed configuration, the prosthesis comprising:
a frame including a plurality of side openings; and
a skirt coupled to an interior of the frame and at least partially covering at least one side opening of the plurality of side openings,
wherein the skirt comprises a pressure-relief arrangement configured to prevent or restrict billowing of an area of the skirt extending over the at least one side opening, and
wherein the pressure relief arrangement comprises at least one fluid passage through the area of the skirt at least partially covering the at least one side opening.

2. The prosthesis according to claim 1, wherein the at least one fluid passage comprises at least one aperture extending through the skirt.

3. The prosthesis according to claim 1, wherein the at least one fluid passage is configured to permit fluid to flow in a radially inward direction through the skirt and to block fluid flow in a radially outward direction.

4. The prosthesis according to claim 1, wherein the pressure relief arrangement comprises a moveable member, e.g. a moveable flap, associated with the at least one fluid passage and configured and arranged to selectively close the at least one fluid passage to prevent fluid flow through the skirt in a radially outward direction.

5. The prosthesis according to claim 1, wherein the skirt comprises a fabric material configured to prevent liquid from flowing therethrough, and wherein the at least one fluid passage is defined by a region where the fabric material is configured to coney fluid through the skirt in a radially inward direction.

6. The prosthesis according to claim 1, wherein the skirt comprises a hydrophobic, for example superhydrophobic, fabric material configured to prevent liquid from flowing therethrough, and wherein the at least one fluid passage is defined by a region where the fabric material comprises a reduced hydrophobicity relative to the hydrophobic fabric that is configured convey fluid through the skirt in a radially inward direction, optionally wherein the at last one fluid flow channel is defined by a region containing a reduced number of hydrophobic groups.

7. The prosthesis according to claim 6, wherein the region is defined by an area of the fabric of increased wettability relative to the fabric material.

8. The prosthesis according to claim 6, wherein the region comprises a gradient in the hydrophobicity of the fabric material such that the hydrophobicity of the fabric reduces from a radially outer side to a radially inner side of the fabric material.

9. The prosthesis according to claim 6, wherein the at least one fluid passage is configured to permit directional transport through the skirt in a radially inward direction, and to prevent flow through the at least one fluid passage in a radially outward direction.

10. The prosthesis according to claim 1, wherein the skirt comprises a substantially tubular body and first and second substantially circular edges, and wherein the first and/or second edge comprises at least one region defining a curved recessed edge.

11. The prosthesis according to claim 10, wherein the at least one fluid passage comprises at least one aperture extending through the skirt, said at least one aperture positioned at or adjacent to the curved recessed edge.

12. The prosthesis according to claim 1, wherein the frame comprises a plurality of crowns and a plurality of struts with each crown being formed between a pair of opposing struts, and wherein the plurality of side openings are defined by the plurality of crowns and the plurality of struts.

13. The prosthesis according to claim 1, wherein the frame comprises a row of side openings extending around a perimeter thereof, and wherein the row comprises less than twelve side openings, optionally less than ten side openings, for example in the range four to ten side openings, for example in the range six to nine side openings.

14. The prosthesis according to claim 1, wherein the skirt is coupled to an inner surface or an outer surface of the frame, and wherein the skirt extends over fewer than all of the plurality of side openings of the frame.

15. The prosthesis according to claim 1, wherein the frame is formed from a self-expanding material comprising a shape memory material, for example nitinol.

16. The prosthesis according to claim 1, wherein the frame comprises an inner frame and an outer frame, and wherein the plurality of side openings comprises a plurality of side openings on the inner frame and the skirt comprises an inner skirt coupled to the inner frame.

17. The prosthesis according to claim 16, wherein the outer frame is connected to the inner frame and radially surrounds the inner frame, when the prosthesis is in the radially expanded configuration.

18. The prosthesis according to claim 16, wherein the plurality of side openings comprises a plurality of side openings on the outer frame and the skirt comprises an outer skirt coupled to the outer frame.

19. The prosthesis according to claim 1, wherein the prosthesis is a heart valve prosthesis comprising a prosthetic valve component disposed within and secured to the inner frame, the prosthetic valve being configured to prevent blood flow in one direction to convey blood flow through a central lumen of the inner frame.

20. The prosthesis according to claim 19, wherein the heart valve prosthesis is configured for placement within a mitral heart valve or tricuspid heart valve in situ.
